(19) United States
(12) Patent Application Publication
(10) Pub. No.: US 2017/0304582 A1
(43) Pub. Date: Oct. 26, 2017

(54) SYSTEMS FOR BRAIN STIMULATION DURING SLEEP AND METHODS OF USE THEREOF

(71) Applicant: ABLE CEREBRAL, LLC, Ephrata, PA (US)

(72) Inventor: Jun Xia, York, PA (US)

(73) Assignee: ABLE CEREBRAL, LLC, Ephrata, PA (US)

(21) Appl. No.: 15/521,274

(22) PCT Filed: Oct. 20, 2015
(86) PCT No.: PCT/US15/56537

§ 371 Date: Apr. 21, 2017

Related U.S. Application Data
(60) Provisional application No. 61/122,457, filed on Dec. 15, 2008.
(60) Provisional application No. 62/123,328, filed on Nov. 14, 2014.

(51) Int. Cl. A61M 21/00

(52) U.S. Cl. CPC A61M 21/00; A61M 2230/18; A61M 2230/10; A61M 2021/0066; A61M 2021/0016; A61M 2021/0027; A61M 2021/0022; A61M 2021/0044

(57) ABSTRACT

The present invention relates to systems for brain stimulation during sleep and methods for increasing vividness of a subject's dreams or for directing the subject matter of a subject's dreams. The system comprises a brain stimulation module and at least one of a brain energy source and/or a hypnotic source. The methods involve administering to a subject a brain energy molecule and/or a hypnotic and providing to the subject during restorative sleep a sensory stimulation.

### CROSS REFERENCE TO RELATED APPLICATIONS

This document claims priority to and the benefit of U.S. Provisional Patent Application No. 62/122,457 to Jun Xia entitled “A memory stimulation and recovery system” which was filed on Oct. 21, 2014 and U.S. Provisional Patent Application No. 62/123,328 to Jun Xia entitled “Timed release of combination compounds for prevention and therapy of central nerve system energy deficiency symptoms” which was filed on Nov. 14, 2014; the disclosure of which are hereby incorporated herein by reference thereto.

### FIELD OF THE INVENTION

This invention relates to systems and methods of brain stimulation during sleep, including directing the subject matter and/or increasing the vividness of a dream.

### BACKGROUND OF THE INVENTION

While a subject is often deceptively calm during sleep, sleep is in fact a dynamic physiological state where the brain is progressing through alternating cycles of light and deep sleep (or sleep stages). These alternating sleep stages allow the brain and body to physically and cognitively restore itself.

As defined by the American Academy of Sleep Medicine, sleep can be characterized into four stages based on the emitted brain waves. Stage 1 (also known as Stage NREM1 or Stage N1) and Stage 2 (also known as Stage NREM2 or Stage N2) are part of non-rapid eye movement (NREM) sleep. Stage 1 takes place when a person is first falling asleep, and the characteristic brain wave is alpha waves. Stage 2 features transitions between wakefulness and deeper sleep, and the characteristic brain wave is theta waves. Stage 3 (also known as Stage N3) and Stage REM (rapid eye movement sleep) are periods of restorative sleep. Stage 3 is also known as slow-wave sleep (SW), characterized by delta waves, and is associated with stabilized metabolic levels. For example, it has been found that during Stage 3, the levels of glucose, testosterone, and human growth hormone has been stabilized. Stage 3 is associated with overall physical restoration. As named, the fourth stage of sleep features rapid eye movement. Brain waves emitted during Stage REM are rapid low-voltage EEG (electroencephalogram) that is similar to the EEG of a person that is awake. This is stage of sleep in which a person dreams. Stage 4 is associated with cognitive restoration, along with cellular regeneration, memory allocation, and memory retention.

A single sleep cycle begins with Stage 1, then Stage 2 to Stage 3, followed by Stage 2 and then Stage REM. This sleep cycle repeats until the person awakens. On average, an adult human progresses through a single sleep cycle every 2 hours. The time distribution of each stage during a sleep cycle varies through a night of sleep. For example, more Stage 3 sleep is found earlier in the sleep period, such as in the evening, while more Stage REM sleep is found later in the sleep period, such as the morning hours.

While sleep has been used to study brain function and current therapeutics are directed towards improving the quality of sleep, the period of sleep itself has not been used as a therapeutic. Until this present application, the dynamic brain activity of sleep has not been taken advantage of for improving the neurological health.

### BRIEF SUMMARY OF THE INVENTION

In one embodiment, the invention is directed to a system for brain stimulation during sleep. The system comprises a brain stimulation module comprising a sensory stimulation unit, a recording unit, wherein the recording unit detects and records the stages of sleep, and a program to interpret the brain electrical activity as detected by the recording unit and direct the activation of the sensory stimulation unit. The program interprets the information from the recording unit so when the recording unit detects the subject is in restorative sleep, the program instructs the sensory stimulation unit to deliver sensory stimulation to the subject. The sensory stimulation may be at least one of sound, scent, taste, tactile, and visual stimulation. In some embodiments, the recording unit detects brain waves and records electroencephalography. In other embodiments, recording unit detects brain waves and records electroencephalography. In some implementations, restorative sleep is Stage 3 sleep, Stage REM sleep or indicated by the recording unit detecting rapid eye movements, delta waves, or rapid low-voltage EEG similar to when a person is awake.

The system further comprises at least one of a brain energy supply source or a hypnotic source. The brain energy supply source comprises a brain energy molecule while the hypnotic source comprises a hypnotic. In preferred embodiments, the brain energy supply source comprises the brain energy molecule in a delayed and sustained release formulation. The hypnotic source may comprise the hypnotic in a delayed and sustained release formulation, an immediate release formulation, or sustained release formulation. The delayed and sustained release formulation is a formulation configured to release less than 15% by weight of the brain energy molecule and/or the hypnotic within 2 hours after administration. Preferably, less than 60% of the brain energy molecule and/or the hypnotic is released within the first 4 hours after administration, at least 80% of the brain energy molecule and/or the hypnotic is released within 8 hours after administration, and the brain energy molecule and/or the hypnotic is released at a sustained rate 2 hours after administration. The hypnotic source may also comprise the hypnotic in an immediate release formulation.

Another embodiment of the invention is directed to methods of increasing vividness of a subject's dreams. The methods comprise administering to the subject, prior to the subject enters a period of sleep, a brain energy molecule in a delayed and sustained release formulation and a hypnotic. The hypnotic may be in a sustained release, immediate release, and/or a delayed and sustained release formulation. The methods may further comprise stimulating the subject with at least one sensory stimulation during restorative sleep.

A third embodiment of the invention is directed to methods of directing the subject matter of a subject's dreams. The methods comprise administering to the subject, prior to the subject enters a period of sleep, a brain energy molecule in a delayed and sustained release formulation and stimulating the subject with at least one sensory stimulation during restorative sleep. The methods may further comprise administering to the subject a hypnotic prior to the subject enters a period of sleep. The hypnotic may be in a sustained release, immediate release, and/or a delayed and sustained release formulation. In some embodiments, the methods further comprise detecting brain activity of the subject and determining from the brain activity of the subject the subject is in restorative sleep.

For the methods of the invention, a delayed and sustained release formulation is a formulation configured to release less than 15% by weight of the brain energy molecule and/or the hypnotic within 2 hours after administration. Preferably, less than 60% of the brain energy molecule and/or the hypnotic is released within the first 4 hours after administration, at least 80% of the brain energy molecule and/or the hypnotic is released within 8 hours after administration, and the brain energy molecule and/or the hypnotic is released at a sustained rate 2 hours after administration. The sensory stimulation according to the methods of the invention may be sound, scent, taste, tactile, or visual, and restorative sleep may be Stage 3 sleep, Stage REM sleep, or a combination thereof. Restorative sleep may be detected by recording the electrical activity of the subject's brain or measuring movement of the subject's eyes. For the methods of the invention, the subject may be continuously stimulated with the sensory stimulation for the duration of restorative sleep. In some implementations, the subject may be continuously stimulated with the sensory stimulation for the duration of restorative sleep during the course of a period of sleep.

The brain energy molecule described in the systems and methods of the invention may be is selected from the group consisting of: glucose, mannose, lactic acid (lactate), and pyruvic acid (pyruvate). The hypnotic described in the systems and methods of the invention may be selected from the group consisting of: a z-drug, a benzodiazepine, a barbiturate, an antidepressant, and a natural sleep medication. The antidepressant may be mirtazapine. The natural sleep medication may be melatonin, valerian, hops, or derivatives thereof.

### DETAILED DESCRIPTION OF THE INVENTION

Embodiments and applications of the invention presented here are described below. Unless specifically noted, it is intended that the words and phrases in the specification and the claims be given their plain, ordinary, and accustomed meaning to those of ordinary skill in the applicable arts.

As used herein, the term “sleep” refers to a physiological state of relative unconsciousness and inaction of the voluntary muscles. Sleep comprises different stages. Stages of sleep may be defined by EEG characterizations—such as delta waves. Sleep may also be characterized by depth (light or deep), by physiological characteristics (REM or NREM), or by anatomic level (e.g. pontine, mesencephalic, rhombencephalic, rolandic, etc.).

As used herein, the term “dream” refers to a mental activity during sleep in which events, thoughts, emotions, and images are experienced as real.

The present invention is related to the discovery that brain stimulation during sleep can direct the subject matter of dreams and to increase the vividness of dreams. Brain stimulation may be external stimulation or enhanced potential for endogenous brain stimulation. External stimulation may be caused by sensory stimulation. The sensory stimulation may be sound, smell, taste, touch, and visual. Enhanced potential for endogenous brain stimulation may be caused by greater potential for brain activity, for example, by providing a critical level of blood sugar level.

In one aspect, the present invention is directed to a system for brain stimulation during sleep. Though certain brain stimulation has been used to study the correlation between sleep stages and memory consolidation, the present invention is the first to disclose that providing brain stimulation to a sleeping subject may be used to enhance the vividness of dreams as well as direct the subject matter of the dreams. With increased vividness of dreams, the subject is more able to retain the content of dreams. In some implementations, the brain stimulation may be used to aid remembrance of past experiences. For example, the increased vividness of the related dream facilitates memorization of the dream contents so that the memory of the past experience may be recovered. Thus the system may be used for brain training. In some instances, system may inhibit memory loss due to dementia, for example Alzheimer's disease. The system may also inhibit mild cognitive impairment. The system comprises at least one of the following components: a brain energy supply source, a hypnotic, and a brain stimulation module. In preferred embodiments, the system comprises at the brain stimulation module.

In another aspect, the present invention is directed to methods of increasing vividness of a subject's dreams. The methods comprise administering to the subject, prior to the subject enters a period of sleep, a brain energy molecule and/or a hypnotic. The method may further comprise stimulating the subject with sensory stimulation when the subject is in restorative sleep.

In still another aspect, the present invention is directed to methods of directing the subject matter of a subject's dreams. The methods comprise administering to the subject a brain energy molecule in a delayed and sustained release formulation and stimulating the subject stimulating the subject with sensory stimulation when the subject is in restorative sleep. The methods may further comprise detecting brain activity of the subject and determining from the brain activity of the subject the subject is in restorative sleep. In some implementations, the methods comprise administering to the subject a hypnotic. The hypnotic may be in an immediate release formulation or a delayed and sustained release formulation. In some implementation, the brain activity of the subject is detected by recording the electrical activity of the subject's brain or by measuring movement of the subject's eyes.

For both methods of directing the subject matter of a subject's dreams and methods of increasing vividness of a subject's dreams, restorative sleep is Stage 3, Stage REM, or both. In preferred embodiment, the brain energy molecule is in a delayed and sustained release formulation. The hypnotic may also be in a delayed and sustained release formulation, or the hypnotic may be in a sustained release formulation. Preferably, a hypnotic with a short half-life (for example zolpidem) is in a formulation for delayed release and sustained release while a hypnotic with a long half-life (for example mirtazapine) is in a sustained release or immediate release formulation. The brain energy molecule and the hypnotic may be administered separately or together. In some embodiments, a second administration of the hypnotic may be needed while the subject is asleep.

The sensory stimulation may be sound, scent, taste, tactile, visual, or a combination thereof. The sensory stimulation may be delivered by the brain stimulation module described below.

### 1. THE BRAIN STIMULATION MODULE

The brain stimulation module comprises a recording unit, a sensory stimulation unit, and a program. The recording unit monitors and tracks a subject's brain activity. In some embodiments, the recording unit monitors the EEG of a subject. The sensory stimulation unit provides sensory stimulation to the subject. The sensory stimulation may be by sound, smell, taste, touch, or visual. Thus the sensory stimulation unit delivers stimulation by at least one of sound, scent, taste, tactile, and visual. The program interprets the data from the recording unit to provide instructions to the sensory stimulation unit. Specifically, the program instructs the sensory stimulation when to delivery the stimulation. In some implementations, the program may be an application, for example an application for mobile devices. In some embodiments, the program can control the strength and/or duration of the stimulation. For example, in some implementations, the program directs the sensory stimulation unit to provide sensory stimulation to the subject when he or she has entered restorative sleep. Specifically, the system may provide sensory stimulation to the subject once he or she has entered Stage 3 or Stage REM sleep. The program also controls the extent and period by ensuring that the stimulation is only provided during desired stage of sleep. In the case the recording unit indicates the sensory stimulation is prematurely moving the subject into wakefulness, the program may halt the sensory stimulation and/or adjust the intensity of the sensory stimulation so that the sensory stimulation does not disturb the quality of the subject's sleep.

### a. Sound Stimulation

For sound stimulation, the sensory stimulation unit comprises a sound delivery device and prerecorded sounds. The sound delivery device transmits the prerecorded sounds to the subject. Thus in some implementations, the sound delivery device is a portable media player. Non-limiting examples of the sound delivery device includes a tape recorder, a CD player, a MP3 player, or a smartphone. The sound delivery device may further comprise speakers. In some embodiments, where the tape recorder, CD player, or MP3 player lacks its own speakers, the speakers provide the sound stimulation to the subject. In other embodiments where the tape recorder, CD player, or MP3 player has its own speakers, the speakers are a separate system adjusted for delivery sound stimulation to a sleeping subject. The prerecorded sound can be music pieces of the subject personal favorite during his or her life, or the voices of the subject's families, friends or colleagues. The prerecorded sounds may also be sound related to general aspects of an active life, for example the sound of airplanes, vehicles, machinery, animals, or any related sound sources. To facilitated recall of a past event, the prerecorded sound may be audio from a recording of the past event.

### b. Scent Stimulation

For scent stimulation, the sensory stimulation unit comprises a reservoir for housing a fragrance product (for example perfume or aftershave), scented oil (for example perfume oils or essential oils), or manmade chemical having a scent. The sensory stimulation unit for smell may further comprise additional components for delivery of the smell to the subject, for example, tubing and fans. In preferred embodiments, scent stimulation provides to the subject a smell that he or she is familiar with or enjoys.

### c. Taste Stimulation

For taste stimulation, the stimulation unit comprises a fluid reservoir. The fluid reservoir houses liquids such as any liquid that possess a taste, for example, juices and liquid flavoring agent. The sensory stimulation unit for smell may further comprise additional components for delivery of the fluids to the subject, for example, a feeding tubing and pumping motor. In preferred embodiments, taste stimulation provides to the subject a taste that he or she is familiar with or enjoys.

### d. Tactile Stimulation

For tactile stimulation, the stimulation unit comprises an apparatus that touches the subject. For example, the apparatus may provide only come into physical contact with the subject or provide the subject with a massage. The apparatus may also be capable of changing the temperature around the subject's body. Thus the apparatus may further comprise heating or cooling fans.

### e. Visual Stimulation

For visual stimulation, the stimulation unit comprises a color-projecting device. The color-projecting device alters the subject's perception of the color of its environment. For example, the color-projecting device may project a light, colored or uncolored, at the subject. In some embodiments, the color-projecting device shines the light onto the subject's eyelids. On the other hand, the color-projecting device may control the background colors of the environment by projecting a light, colored or uncolored, into the environment. In some implementations, the color-projecting device may project light at the subject and at the environment.

### 2. THE BRAIN ENERGY SUPPLY AND THE HYPNOTIC

The inventor of this patent application observed that to achieve quality sleep with the aid of medication also requires a certain brain energy supply during sleep. Further, the brain energy supply during sleep has to maintain certain level to achieve valuable dream activities, which not only can benefit general human health, but also may prevent and reverse early stage of central nerve system degenerative diseases such as Alzheimer's disease and dementia.

The brain energy supply source comprises brain energy molecules formulated for delayed and sustained release. Non-limiting examples of the brain energy molecule glucose, mannose, lactic acid (lactate), and pyruvic acid (pyruvate). The delayed and sustained release formulation of the brain energy molecule may be in the form of tablets, capsules, suppositories, transdermal or trans-buccal devices, or IV drips. This component is essential if the person shows hypoglycemia during sleep, or additional brain energy is warranted to induce brain activities such as certain brain waves for memory recovery. In some embodiments, the brain energy supply source or the delayed and sustain release formulation comprises about 250 mg and about 2500 mg, about 250 mg to about 1750 mg, or about 250 mg to about 1250 mg of the brain energy molecule. In other embodiments, the brain energy supply source or the delayed and sustain release formulation comprises between about 500 mg and about 1000 mg, about 500 mg to about 1250 mg, about 500 mg to about 1750 mg, or about 500 mg to 2500 mg of the brain energy molecule. In still other embodiments, the brain energy supply source or the delayed and sustain release formulation comprises about 750 mg of the brain energy molecule.

The hypnotic may be used for ensuring adequate sleeping time to ensure the subject does not have a sleep deficit during the period of sleep. In some implementations, the hypnotic may be used to ensure the subject's brain is capable of responding to the sensory stimulation during restorative sleep. The hypnotic may be a z-drug, a benzodiazepine, a barbiturate, an antidepressant, or a natural sleep medication. Specifically, the antidepressant may be mirtazapine. The natural sleep medication may be melatonin, valerian, hops, or derivatives thereof The amount of the hypnotic administered to the subject is determined by the customary dosing for the hypnotic. Accordingly, the hypnotic source comprises the customary single dose for the hypnotic or at least the amount of the customary single dose.

In some implementations, the hypnotic and brain energy molecule are administered so that the subject absorbs the hypnotic and brain energy molecule at the same time. Accordingly, the hypnotic and brain energy may be mixed in a single composition that is administered to the subject. Preferably, the hypnotic and the brain energy molecule are uniformly mixed in the single composition. Alternatively, the hypnotic and the brain energy molecule may be premixed to produce two portions. In some embodiments, a first portion comprises just the hypnotic while a second portion comprises the hypnotic with the brain energy molecule. In some aspects, the first portion is the hypnotic in a delayed release formulation. In other aspects, the first portion is the hypnotic in an immediate release formulation where the total amount is designed to release immediately upon administration. Immediately release of the hypnotic is appropriate where the hypnotic has a long half-life.

In embodiments where the hypnotic and brain energy molecule are premixed in a solid dosage form comprising two portions with the hypnotic being formulated for immediate release, the first portion may be the outer layer of the second portion. In some embodiments, the first portion may comprise beads of the hypnotic with disintegration agents for rapid dissolution upon taken at bedtime.

### a. Delayed Sustained Release

The delayed and sustained release formulation is configured to release less than 15% (e.g., less than 10%, 5%, and 2%), by weight, of a target molecule within 2 hours after administration. After 2 hours, the target molecule is preferably released at a sustained rate such that less than 60% (e.g., less than 55%, 50%, 40%), by weight, of the target molecule is released within the first 4 hours of administration, and at least 80% (e.g., at least 85%, 90%, or 95%), by weight, of the target molecule is released within 8 hours after administration. In a more preferred embodiment, less than 5%, by weight, of the target molecule is released within 2 hours after administration, less than 55% of the target molecule is released within the first 4 hours and at least 85%, by weight, of the target molecule is released within 8 hours after administration.

In some embodiments, delayed and sustained release formulation preferably comprises a transdermal preparation for certain transdermal embodiments. The transdermal preparation typically includes a skin permeation enhancer formulation. A preferred embodiment of skin permeation enhancer formulation comprises at least one glycol, monothioglycerol, at least one of 2-methyl-3-hydroxypyranone or 2-ethyl-3-hydroxypyranone and an aliphatic carboxylic acid of 8 to 24 carbon atoms or an ester of said acid with an aliphatic alcohol of 1 to 14 carbon atoms and 1 to 2 hydroxy groups. In a more preferred embodiment, the skin permeation enhancer formulation has a composition of 10% to 95%, by weight, of the at least one glycol, 1% to 10%, by weight, of monothioglycerol, 2% to 30%, by weight, of the at least one of 2-methyl-3-hydroxypyranone or 2-ethyl-3-hydroxypyranone and 2%-10%, by weight, of the aliphatic carboxylic acid of 8 to 24 carbon atoms or an ester of said acid with an aliphatic alcohol of 1 to 14 carbon atoms and 1 to 2 hydroxy groups. The at least one glycol is typically selected from the group consisting of propylene glycol, butylenes glycol, hexylene glycol, ethoxydiglycol, dipropylene glycol and pentylene glycol. In a preferred embodiment, the skin permeation enhancer formulation has a composition of about 70-80%, by weight, butylene glycol, about 3-9%, by weight, monothioglycerol, about 10%, by weight, 2-methyl 3-hydroxy pyranones and about 4-12%, by weight, oleic acid. In a most preferred embodiment, the skin permeation enhancer formulation has a composition of about 76%, by weight, butylene glycol, about 6%, by weight, monothioglycerol, about 10%, by weight, 2-methyl 3-hydroxy pyranones and about 8%, by weight, oleic acid.

In some embodiment, a transdermal delivery device facilitated delayed and sustained delivery of a compound. The transdermal delivery device preferably comprises a reservoir layer, an adhesive layer, a backing layer and a release liner. The reservoir layer typically comprises absorbent materials inert to chemicals and preferably contains a composition comprising an energy molecule and a skin permeation enhancer formulation.

The adhesive layer is typically attached to the reservoir layer to secure and seal the device to the skin to prevent leaking. In order to allow the adhesive layer to secure and seal the device to the skin, the adhesive layer preferably has margins that extend farther than the reservoir layer to prevent leaking when the device is in use. Preferably, the backing layer is coated by the adhesive layer and, in a preferred embodiment, is impermeable to the energy molecule and/or the hypnotic. In a preferred embodiment, the release liner is inert to chemicals, and the transdermal delivery device is configured to release the composition contained in the reservoir layer such that less than 10% (e.g., less than 5%) by weight of the brain energy molecule or hypnotic is released within 2 hours after administration, the brain energy molecule or hypnotic being released at a sustained rate after 2 hours for 8 hours or more. In some embodiments, the release liner is a sheet of plastic non-permeable film to protect the release of content before applying the transdermal delivery device to the subject.

The backing layer preferably comprises any material that is impermeable for the brain energy molecule and/or hypnotic and physically and chemically stable to the skin permeation enhancer formulation. In a preferred embodiment, the backing layer is comprised of a commercially available material, such as SCOTCHPAK by 3M, though other materials may be utilized. In other embodiments, the adhesive layer is coated to the backing and provides attachment for the reservoir and also surrounds and seals the reservoir onto the skin. The adhesive layer typically comprises any adhesive material that is physically and chemically compatible with the reservoir layer. In a preferred embodiment, the adhesive layer comprises EUDRAGIT acrylic adhesives. In another embodiment, the adhesive layer comprises NATIONAL STARCH acrylic adhesives. Other suitable adhesives may be used as well.

The reservoir layer typically comprises any absorbent material inert to the brain energy molecule and/or hypnotic and the skin permeation enhancer formulation. The absorbent material is fixed to the transdermal delivery device through adhesion to the adhesive layer on the backing layer. In a preferred embodiment, cotton fabric is utilized as the absorbent material. In another embodiment, polypropylene non-woven material is utilized as the absorbent material. Other absorbent materials may also be utilized in addition to these two options.

In a preferred embodiment, the transdermal delivery device is placed anywhere on the subject's skin immediately prior to the subject going to sleep. In a more preferred embodiment, the transdermal delivery device is placed anywhere on the subject's neck immediately prior to going to sleep. In a most preferred embodiment, the transdermal delivery device is placed on the subject's neck area proximate to the subject's carotid artery immediately prior to going to sleep.

In a preferred implementation of this method, the device is configured to load the composition into the reservoir layer after detaching the release liner and before applying the device to the skin. In such an embodiment, there may be a kit utilized which comprises a transdermal delivery device and a bottle of a composition comprising the energy molecule and skin permeation enhancer formulation. A subject detaches the release liner, fills the reservoir layer with the composition from the bottle, and then applies the device to the subject's neck proximate to the subject's carotid artery. In another implementation of this method, the reservoir layer is preloaded with the liquid mixture of energy molecules and enhancers.

In certain embodiments, the delayed and sustained release formulation is in the form of an oral tablet. Other suitable oral dosage forms include capsules and caplets. Preferably, the capsules are designed to suitable sizes or compositions based on the age or physical characteristics of the patient as well as the severity of the disease. Such dosage forms are prepared using conventional methods known to those in the field of pharmaceutical formulation and described in the pertinent texts, (e.g., Remington: The Science and Practice of Pharmacy, Twentieth Ed. (Philadelphia, Pa.: Lippincott Williams & Wilkins, 2000)). Tablets and capsules represent the most convenient oral dosage forms, in which case solid pharmaceutical carriers are employed.

Tablets may be manufactured using standard tablet processing procedures and equipment. One method for forming tablets is by direct compression of a powdered, crystalline or granular composition containing the active agent(s), alone or in combination with one or more carriers, additives, or the like. As an alternative to direct compression, tablets can be prepared using wet-granulation or dry-granulation processes. Tablets may also be molded rather than compressed, starting with a moist or otherwise tractable material; however, compression and granulation techniques are preferred.

In those embodiments, wherein the dosage form is a capsule, the brain energy molecule and/or hypnotic is typically encapsulated in the form of a solid (including particulates such as granules, beads, powders or pellets). Suitable capsules may be either hard or soft, and are generally made of gelatin, starch, or a cellulosic material, with gelatin capsules preferred. Two-piece hard gelatin capsules are preferably sealed, such as with gelatin bands or the like. (See, for e.g., Remington: The Science and Practice of Pharmacy, supra), which describes materials and methods for preparing encapsulated pharmaceuticals.

Preferred solid dosage forms, whether tablets, capsules, caplets, or particulates, are preferably coated or have a coating so as to provide for delayed and sustained release. Dosage forms with delayed and sustained release coatings may be manufactured using standard coating procedures and equipment. (See, for e.g., Remington: The Science and Practice of Pharmacy, supra).

In a preferred embodiment, the oral tablet or capsules comprise a coating of a pH-dependent polymer. The pH-dependent polymer is preferably selected from the group consisting of: a polyacrylate material, a cellulose acetate phthalate, cellulose phthalate hydroxyl propyl methyl ether, polyvinyl acetate phthalate, hydroxyl propyl methyl cellulose acetate succinate, cellulose acetate trimellitate and shellac. In another preferred embodiment, the oral tablet further comprises a hydrophilic polymer. The hydrophilic polymer is preferably selected from the group consisting of: hydroxy propyl methylcellulose, hydroxypropyl ethylcellulose, hydroxypropyl cellulose, hydroxy ethylcellulose, methylcellulose, xantham gums, alginate salts, polyethylene oxide, carboxyvinyl polymer, and a salt of a carboxymethyl cellulose. The hydrophilic polymer preferably has a viscosity within the range of from about 60 to about 7,000,000 centipoises in a 2% by weight water solution at 25 degrees Celsius, as measured by a Brookfield LV viscometer. In another preferred embodiment, the oral tablet further comprises a water-insoluble polymer. The water-insoluble polymer is preferably selected from the group consisting of: ethyl cellulose, acetate cellulose and polyacrylate copolymer. The coatings provide for the delayed and the sustained release of the energy molecules.

The present invention is further illustrated by the following examples that should not be construed as limiting. The contents of all references, patents, and published patent applications cited throughout this application are incorporated herein by reference in their entirety for all purposes.

### EXAMPLES

### A. Test Results

1. A healthy male adult volunteer was administered 2 caplets of delayed and sustained release glucose at bedtime. The subject reported that he had more vivid dreams with the administration of the delayed and sustained release glucose and was more able to recall past memory upon waking up.
2. A healthy male adult volunteer was treated using the system of the present invention. The subject was connected with brain simulation module and administered 3 caplets of delayed and sustained release glucose at bedtime prior to the subject going the sleep. The sensory stimulation received was sound stimulation during certain brain waves. The prerecorded sound play was a conversation between the subject's family members in a past event. Upon awakening, the subject reported that he could recall dreams related to the voice stimulation content, which expanded his memory of the past event.
3. A male adult volunteer was administered 2 caplets of delayed and sustained release of glucose with sustained release of zolpidem at bedtime. The subject reported that he experienced better sleep and found the dreams he had to be more vivid and memorable.
4. A 54-year-old healthy male subject, without diabetes, took 2 caplets of delayed and sustained release glucose at bedtime every day for one week. Subsequently, the subject 5 mg to 10 mg zolpidem in addition to the 2 caplets of delayed and sustained release glucose at bedtime every day for one week. The subject reported that he had more vivid dreams during both weeks. The subject also reported that he remembered more of the dreams in the mornings during both weeks, but the improvement was most noticeable when he took zolpidem. During the first week, the subject also checked his blood glucose levels at bedtime, between 2 to 3 am, and upon waking up in the morning. The average readings were 80 mg/L, 86 mg/L, and 85 mg/L.
5. A 58-year-old healthy male subject, without diabetes, took 2 caplets of delayed and sustained release of glucose at bedtime. The subject reported having more dreams while remembering more of dreams upon waking up.
6. A 62-year-old male diabetic subject was administered 2 caplets of delayed and sustained release glucose at bedtime every day for a week. For two of these days, the subject also took zolpidem at bedtime. The subject's diabetic treatment comprises taking Repaglinide, an insulin stimulator, 30 minutes before meals. While under this therapy, the subject reported a history of occasional nocturnal hypoglycemia. The subject reported that he had more dreams and remembered the dream when he woke up in the mornings. He found that the beneficial effect on dreams and memory was more pronounced when he also took zolpidem at bed time.
7. A 79-year-old male diabetic subject took 2-3 caplets of delayed and sustained release glucose at bedtime every day for a week. His diabetic treatment comprises taking short and medium acting insulin daily before meals. The subject said that he felt he has dreamt less since he started using the insulin product. After taking the delayed and sustained release glucose at bedtime, the subject reported that he had more dreams during night and recalled then after waking up.

### B. Example Formulations

Formulation 1. Zolpidem (6.25 mg) is mixed with glucose and the other ingredients before tableting for a delayed and sustained release formulation.
Formulation 2. Formulation 1 is film-coated with a composition comprising zolpidem (6.25 mg) and ethylcellulose.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1. A system for brain stimulation during sleep, the system comprising:
at least one of a brain energy supply source and a hypnotic source, wherein the brain energy supply source comprises a brain energy molecule and the hypnotic source comprises a hypnotic;
a sensory stimulation unit;
a recording unit, wherein the recording unit detects and records the stages of sleep; and
a program to interpret records from the recording unit and activate the sensory stimulation unit, wherein the program directs activation of the sensory stimulation unit upon detection by the recording unit of restorative sleep and the sensory stimulation unit delivers at least one sensory stimulation selected from the group consisting of: sound, scent, taste, tactile, and visual.

2. The system of claim 1, wherein the brain energy supply source is configured to release less than 15% by weight of the brain energy molecule within 2 hours after administration; the brain energy supply source further configured to release less than 60% of the brain energy molecule within the first 4 hours after administration and at least 80% of the brain energy molecule within 8 hours after administration, wherein the brain energy molecule is released at a sustained rate 2 hours after administration.

3. The system of claim 1, wherein the hypnotic source is configured to release less than 15% by weight of the hypnotic within 2 hours after administration; the hypnotic source further configured to release less than 60% of the hypnotic within the first 4 hours after administration and at least 80% of the hypnotic within 8 hours after administration, wherein the hypnotic is released at a sustained rate 2 hours after administration.

4. The system of claim 1, wherein the recording unit detects brain waves and records electroencephalography and/or detects and records eye moments.

5. (canceled)

6. The system of claim 1, wherein restorative sleep is Stage 3 sleep and/or Stage REM sleep.

7. The system of claim 1, wherein restorative sleep is indicated by the recording unit detecting delta waves or rapid low-voltage EEG similar to when a person is awake or by the recording unit detecting rapid eye movements.

8. (canceled)

9. The system of claim 1, wherein the program directs activation of the sensory stimulation unit upon the recording unit detecting the start of restorative sleep.

10. The system of claim 1, wherein the sensory stimulation unit delivers sound stimulation.

11. The system of claim 10, wherein the sensory stimulation unit comprises a sound delivery device and prerecorded sounds, wherein the sound delivery device transmits the prerecorded sounds.

12. (canceled)

13. A method of increasing vividness of a subject's dreams, the method comprising:
administering to the subject, prior to the subject enters a period of sleep, a brain energy molecule configured to release less than 15% by weight of the brain energy molecule within 2 hours after administration, less than 60% of the brain energy molecule within the first 4 hours after administration, and at least 80% of the brain energy molecule within 8 hours after administration and configure so that the brain energy molecule is released at a sustained rate 2 hours after administration; and
administering to the subject a hypnotic prior to the subject enters a period of sleep.

14. The method of claim 13, wherein the hypnotic is configured to release less than 15% by weight of the hypnotic within 2 hours after administration, less than 60% of the hypnotic within the first 4 hours after administration, and at least 80% of the hypnotic within 8 hours after administration and configured so that the hypnotic is released at a sustained rate 2 hours after administration.

15. The method of claim 13, further comprising stimulating the subject with at least one sensory stimulation selected from the group consisting of: sound, scent, taste, tactile, and visual when the subject is in restorative sleep.

16. A method of directing the subject matter of a subject's dreams, the method comprising:
administering to the subject a brain energy molecule configured to release less than 60% of the brain energy molecule within the first 4 hours after administration and at least 80% of the brain energy molecule within 8 hours after administration, wherein the brain energy molecule is released at a sustained rate 2 hours after administration prior to the subject goes to sleep; and
stimulating the subject with at least one sensory stimulation selected from the group consisting of: sound, scent, taste, tactile, and visual when the subject is in restorative sleep.

17. The method of claim 16 further comprising administering to the subject a hypnotic prior to the subject enters a period of sleep.

18. The method of claim 17, wherein the hypnotic is configured to release less than 15% by weight of the hypnotic within 2 hours after administration; the medicament further configured to release less than 60% of the hypnotic within the first 4 hours after administration and at least 80% of the hypnotic within 8 hours after administration, wherein the hypnotic is released at a sustained rate 2 hours after administration.

19. The method of claim 15, wherein the subject is continuously stimulated with the at least one sensory stimulation for the duration of restorative sleep.

20. (canceled)

21. The method of claim 13 further comprising:
detecting brain activity of the subject; and
determining from the brain activity of the subject the subject is in restorative sleep.

22. The method of claim 21, wherein detecting brain activity of the subject comprises at least one step selected from the group consisting of: recording the electrical activity of the subject's brain and measuring movement of the subject's eyes.

23. (canceled)

24. The method of claim 22, wherein restorative sleep is selected from the group consisting of: Stage 3, Stage REM, or a combination thereof.

25. The method of claim 13, wherein:
the brain energy molecule is selected from the group consisting of: glucose, mannose, lactic acid (lactate), and pyruvic acid (pyruvate);
wherein the hypnotic is selected from the group consisting of: a z-drug, a benzodiazepine, a barbiturate, an antidepressant, and a natural sleep medication.

26-28. (canceled)
